(19) United States
(12) Patent Application Publication
(10) Pub. No.: US 2008/0152712 A1
(43) Pub. Date: Jun. 26, 2008

(54) RAPIDLY DISINTEGRATING LYOPHILIZED ORAL FORMULATIONS OF A THROMBIN RECEPTOR ANTAGONIST

(76) Inventors: David Monteith, Pittstown, NJ (US); Enrico P. Veltri, Princeton, NJ (US); Srinivas Duggirala, Raritan, NJ (US); Michael Angelo Falvo, Chester, NJ (US); John R. Erbey, Flemington, NJ (US); Kung-I Feng, Basking Ridge, NJ (US); Anastasia Pavlovsky, Morris Plains, NJ (US); Suliman Chawdry, South Plainfield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/860,165

(22) Filed: Sep. 24, 2007

Related U.S. Application Data
(60) Provisional application No. 60/847,306, filed on Sep. 26, 2006.

(51) Int. Cl. A61K 9/00; A61K 31/443; A61P 9/00

(52) U.S. Cl. 424/484; 514/337

(57) ABSTRACT

Disclosed is a lyophilized rapidly disintegrating solid dosage form, one embodiment of which comprises a thrombin receptor antagonist such as,

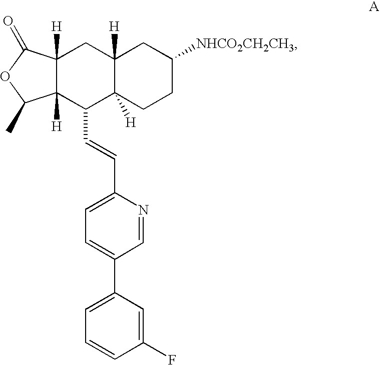

or a pharmaceutically acceptable salt or hydrate thereof, a polymer such as gelatin, and a matrix forming agent such as mannitol. Systems for effectively buffering the pre-lyophilized suspension are taught, along with methods of treating patients at risk for acute coronary syndrome by administering such a rapidly disintegrating solid dosage form.

### CROSS-REFERENCE TO RELATED APPLICATIONS

This application claims the benefit of U.S. provisional application No. 60/847,306, filed Sep. 26, 2006.

### FIELD OF THE INVENTION

The present invention relates to rapidly disintegrating orally administered pharmaceutical compositions containing a thrombin receptor antagonist and their uses in the treatment of patients at risk of acute coronary syndrome.

### BACKGROUND OF THE INVENTION

Thrombin is known to have a variety of activities in different cell types and thrombin receptors are known to be present in such cell types as human platelets, vascular smooth muscle cells, endothelial cells and fibroblasts. It is believed that thrombin receptor antagonists (“TRAs”), also known as protease activated receptor (PAR) antagonists, are useful in the treatment of thrombotic, inflammatory, atherosclerotic and fibroproliferative disorders, as well as other disorders in which thrombin and its receptor play a pathological role. Acute coronary syndrome is one such disorder.

Acute coronary syndrome (“ACS”) is an umbrella term used to cover any of a group of clinical symptoms compatible with acute myocardial ischemia, including unstable angina, and non-ST segment elevation myocardial infarction (“MI”) and ST segment elevation MI. Acute myocardial ischemia is associated with chest pain due to insufficient blood supply to the heart muscle that results from coronary artery disease (also called coronary heart disease). These life-threatening disorders are a major cause of emergency medical care and hospitalization in the United States. Coronary heart disease is the leading cause of death in the United States. Unstable angina and non-ST-segment elevation myocardial infarction are very common manifestations of this disease.

It is not atypical for an ACS patient to arrive at a hospital emergency room unconscious or otherwise unresponsive or incapable of taking direction immediately following an acute cardiac episode. When it is determined that such a patient could benefit from administration of a thrombin receptor antagonist, it can be important to administer a loading dose sufficient to immediately raise the level of the medication in a patient's cardiovascular system to prevent further damage. However, an unresponsive patient may be incapable of swallowing a conventional orally administered, solid dosage form, such as a tablet or capsule. Thus, there exists the need for pharmaceutically acceptable formulations containing a thrombin receptor antagonist, to provide a loading dose of the thrombin receptor antagonist in such a dosage form that it can be quickly and conveniently administered to a patient who may be unresponsive. Such a dosage form could be administered without creating the need to swallow an essentially intact solid tablet, and without the need to administer concomitantly with water to assist in such swallowing of the intact dosage form. Such formulations may be useful in treating the immediate risks associated with ACS.

Rapidly disintegrating dosage forms which are designed to release the active ingredient in the oral cavity are well known and can be used to deliver a wide range of drugs.

Thrombin receptor antagonists have been suggested in the literature as being potentially useful in treating a variety of cardiovascular diseases or conditions including, for example, thrombosis, vascular restenosis, deep venous thrombosis, lung embolism, cerebral infarction, heart disease, disseminated intravascular coagulation syndrome, hypertension (Suzuki, Shuichi, PCT Int. Appls. WO 0288092, WO 0285850 and WO 0285855), arrhythmia, inflammation, angina, stroke, atherosclerosis, ischemic conditions (Zhang, Han-cheng, PCT Int. Appl. WO 0100659, WO 0100657 and WO 0100656).

Thrombin receptor antagonists are disclosed in U.S. Pat. Nos. 6,063,847; 6,326,380; and 6,645,987 and U.S. publication nos. 03/0203927; 04/0216437A1; 04/0152736; and 03/0216437. The use of a small subset of thrombin receptor antagonists to treat a variety of conditions and diseases is disclosed in U.S. publication no. 04/0192753. A crystalline form of the bisulfate salt of a particular thrombin receptor antagonist is disclosed in U.S. Pat. No. 7,235,561. All of these patents and patent publications mentioned herein are incorporated by reference in their entirety.

### SUMMARY OF THE INVENTION

In one embodiment, the invention is directed to a lyophilized rapidly disintegrating solid dosage form comprising an effective amount of a thrombin receptor antagonist. In some embodiments, the thrombin receptor antagonist is selected from the group consisting of:

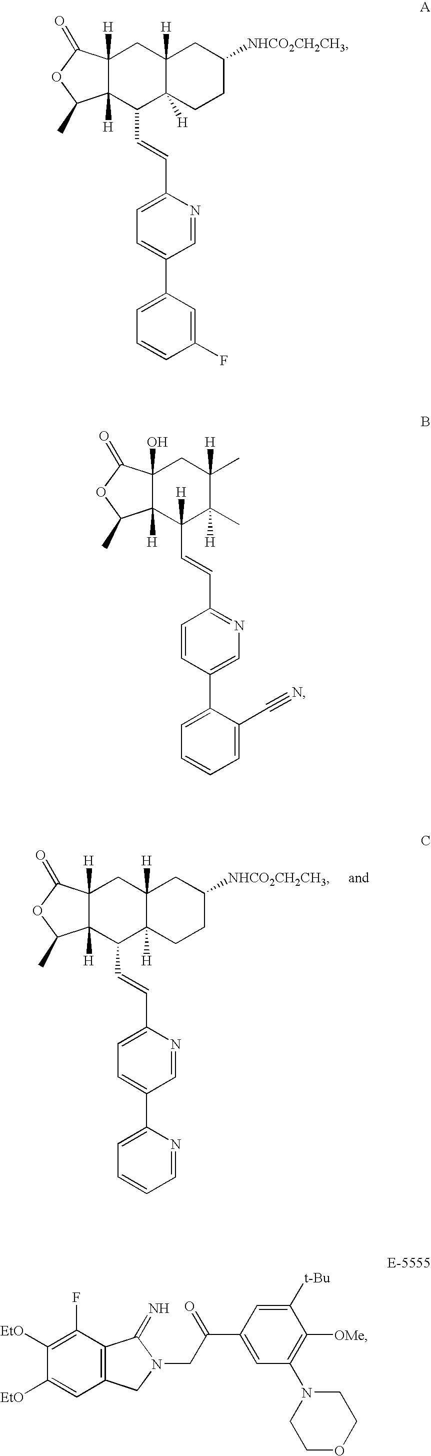

or a pharmaceutically acceptable salt or hydrate thereof.

In some embodiments, the solid dosage form further comprises at least one polymer and at least one matrix forming agent. In some embodiments, the polymer is selected from the group consisting of gelatin, alginates, and modified starches. In some embodiments, the matrix forming agent is selected from the group consisting of mannitol, sorbitol, and dextrins. In some embodiments, the polymer is gelatin and the matrix forming agent is mannitol. In some embodiments, the weight ratio of thrombin receptor antagonist to gelatin is about 2.2 to about 2.3 and the weight ratio of gelatin to mannitol is about 1.0 to about 1.2. In some embodiments, the weight percent of gelatin is about 3.5 on a wet-weight basis. In some embodiments, the weight percent of mannitol is about 3 on a wet-weight basis.

In some embodiments, the rapidly disintegrating dosage form further comprises a buffering system. In some embodiments, the buffering system is selected from the group consisting of acetate, phosphate, and citrate systems.

In some embodiments, an average platelet inhibition of at least about 80% is achieved within 30 minutes of administration.

In some embodiments, the solid dosage form comprises about 20 to about 120 mg of Compound A

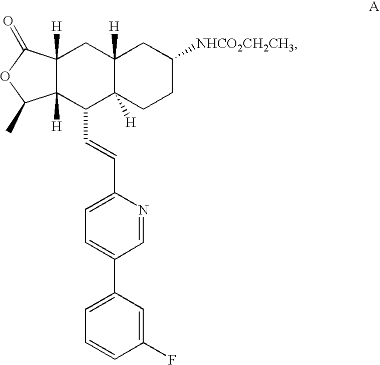

or a pharmaceutically acceptable salt or hydrate thereof.

In some embodiments, the rapidly solid dosage form comprises about 40 mg of Compound A or a pharmaceutically acceptable salt or hydrate thereof. In some embodiments, Compound A is in the form of a bisulfate salt. In some embodiments, the solid dosage form further comprises a polymer, and a matrix forming agent. In some embodiments, the solid dosage form further comprises a buffer system.

In some embodiments, the solid dosage form further comprises gelatin and mannitol. In some embodiments, the solid dosage form comprises about 17.5 mg of gelatin and about 15 mg of mannitol, and a buffer system capable of achieving a pH of between about 3.5 and about 5.5 in the pre-lyophilized suspension as measured directly after addition of said Compound A bisulfate.

In some embodiments, the invention is directed to a lyophilized rapidly disintegrating solid dosage form comprising about 40 mg of Compound A or a pharmaceutically acceptable salt or hydrate thereof, about 18 mg of gelatin, about 15 mg of mannitol, about 19 mg of sodium citrate, and about 8 mg of citric acid.

In some embodiments, the invention is directed to a method of treating a patient at risk of acute coronary syndrome comprising administering any of the rapidly disintegrating solid dosage forms described above.

In some embodiments, the invention is directed to a method of treating a patient at risk of acute coronary syndrome comprising administering to said patient a single lyophilized loading dose comprising an effective amount of a thrombin receptor antagonist and then a series of maintenance doses comprising said thrombin receptor antagonist. In some embodiments, the thrombin receptor antagonist is selected from the group consisting of:

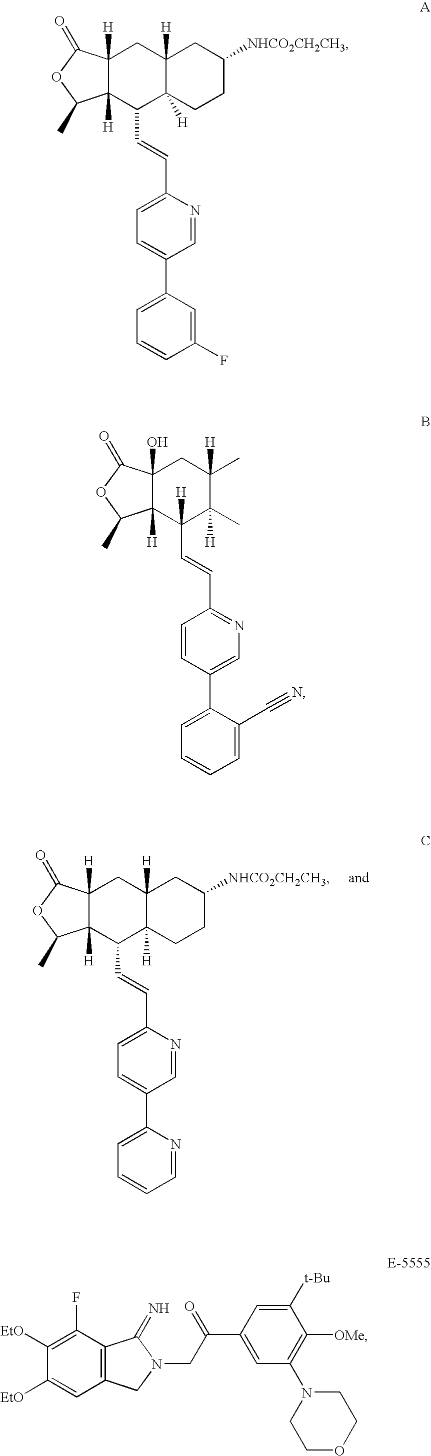

or a pharmaceutically acceptable salt or hydrate thereof. In some embodiments, the thrombin receptor antagonist is

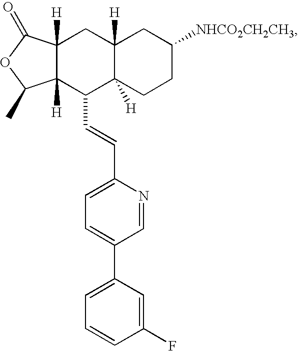

or a pharmaceutically acceptable salt or hydrate thereof. In some embodiments, the thrombin receptor antagonist is in the form of a bisulfate salt. In some embodiments, the loading dose comprises between about 20 and about 120 mg of said thrombin receptor antagonist. In some embodiments, the loading dose comprises about 40 mg of said thrombin receptor antagonist.

### DETAILED DESCRIPTION OF THE INVENTION

A variety of compounds have been demonstrated as displaying activity as thrombin receptor antagonists, many being himbacine analogs. As disclosed in U.S. publication no. 04/0152736, a subset of particularly preferred compounds of Formula I is as follows:

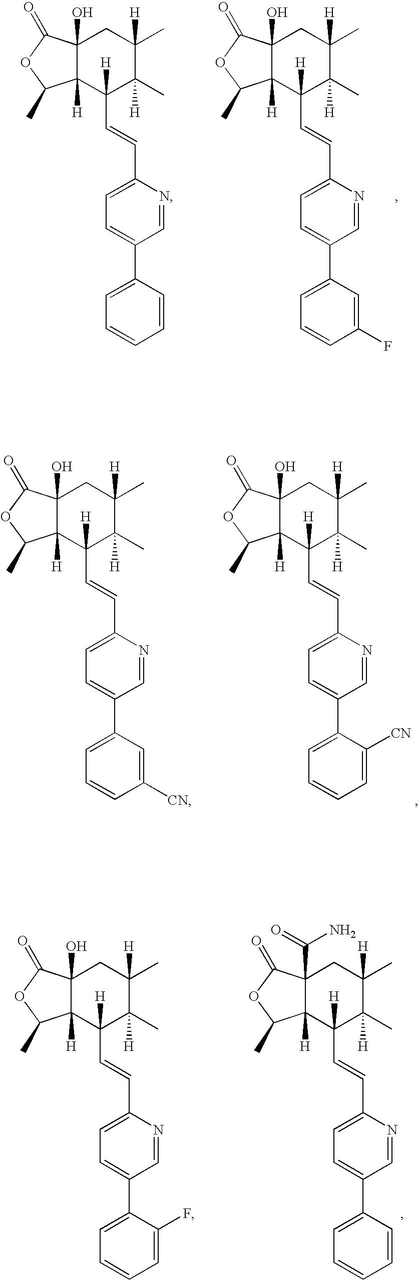

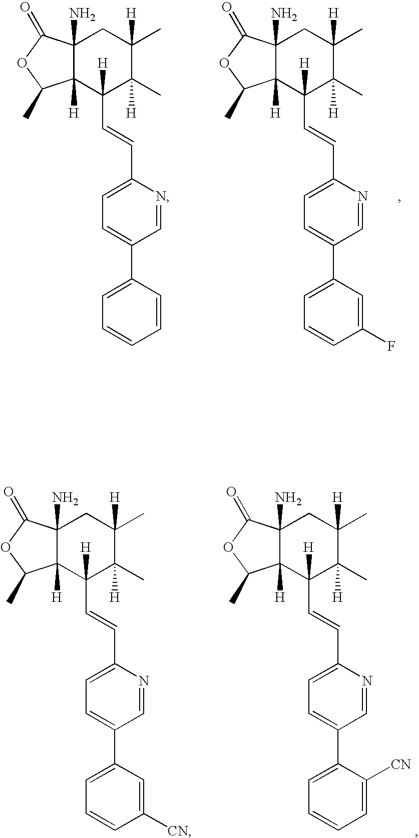

and pharmaceutically acceptable salts thereof.

U.S. publication no. 03/0216437 discloses a subset of thrombin receptor antagonists of Formula II which are both particularly active and selective. These compounds are as follows:

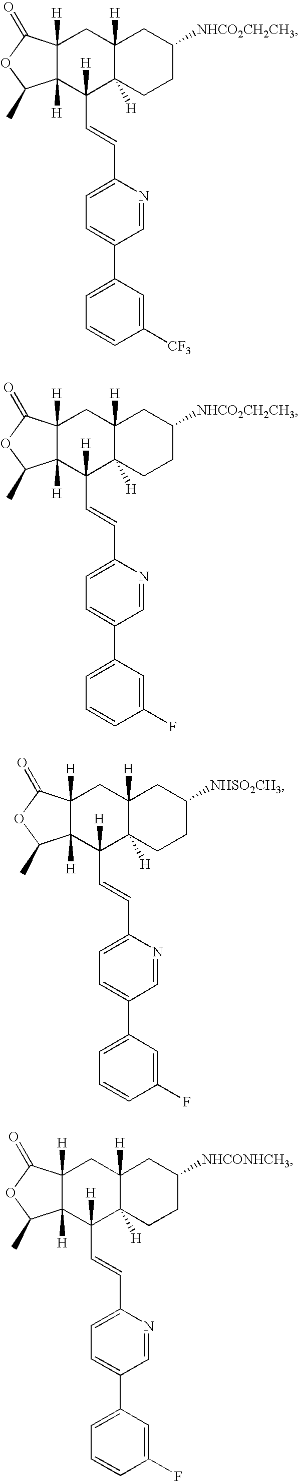

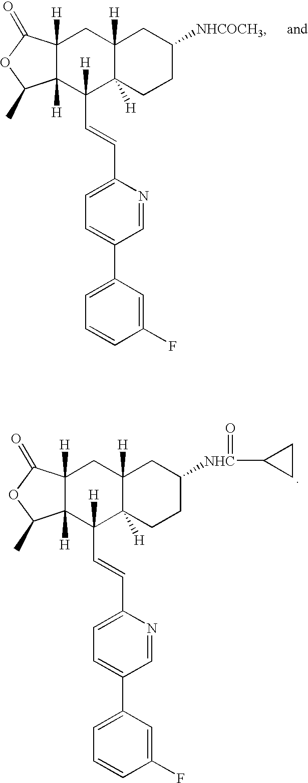

and the pharmaceutically acceptable isomers, salts, solvates and polymorphs thereof.

The following compounds are particularly favored based on their pharmacokinetics and pharmacodynamic characteristics:

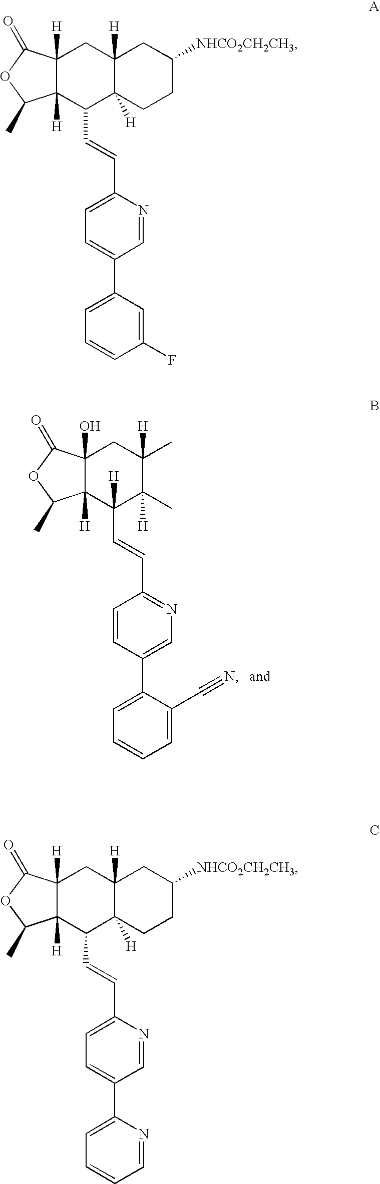

or a pharmaceutically acceptable isomer, salt, hydrate, solvate, polymorphs or co-crystal form thereof. The bisulfate salt of Compound A is currently in development as a thrombin receptor antagonist by Schering-Plough Corp. Its synthesis is disclosed in U.S. publication no. 03/0216437, which publication also discloses Compound C. Compound B is disclosed in U.S. Pat. No. 6,645,987.

Other compounds for use in the formulations of the present invention are disclosed in any of U.S. Pat. Nos. 6,063,847, 6,326,380, U.S. Patent Publications U.S. 03/0203927, U.S. 03/0216437, US 04/0192753, and U.S. 04/0176418, the compound-related disclosures of which are all incorporated by reference in their entirety. Combinations that include other agents that display activity as thrombin receptor antagonists are also within the scope of the present invention, including E5555 currently in development by Eisai, the structure of which is as follows:

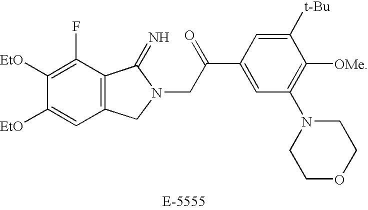

In one embodiment of the present invention, the formulation is an oral solid dosage form that can be swallowed without water, because it disintegrates rapidly on the tongue, in some embodiments, in less than about 60 seconds, preferably, in less than about 30 seconds, more preferably, in less than about 10 seconds, and most preferably, in less than about 3 seconds. Such a rapid disintegration phenomenon may provide for enhanced dissolution of the active ingredient, and subsequent realization of an optimal, i.e., rapid, pharmacokinetic profile of such an ingredient. Preferably, essentially all of the thrombin receptor antagonist dissolves within about 15 minutes.

Dissolution rates of the active ingredients are typically measured in an in vitro setting using pharmaceutical compendial apparatus such as the USP Dissolution Apparatus 1 (basket) or Apparatus 2 (paddle). Alternate dissolution test methodologies may also be employed, e.g., flow-through dissolution cells, based upon the physical nature of the embodiment.

One of the ultimate purposes in providing a rapidly disintegrating solid dosage form is to provide a blood concentration profile of the thrombin receptor antagonist is sufficient to result in a rapid onset of blood platelet inhibition in the patient at risk for ACS. The formulations of the present invention are believed to result in an average platelet inhibition of at least about 80% within 30 minutes of administration. Platelet inhibition is discussed in U.S. Publication no. 03/0216437, which discussion is incorporated herein.

The solid dosage forms of the present invention are in the form of a lyophilized (or freeze-dried) matrix in the shape of a wafer suitable for placement on the tongue. The matrix confers sufficient strength to the dosage form to allow for routine handling during product packaging, storage, and shipment, and to prevent breakage during removal from the package. Once placed within the oral cavity, however, the matrix disintegrates rapidly and may provide for rapid dissolution of the active pharmaceutical agent. Various aspects of lyophilized formulations are disclosed in WO 00/44351.

The matrix is composed of any of one or more of a variety of materials designed to achieve a number of objectives. A polymer can be used to form a glassy amorphous structure which imparts strength and resilience during handling. A matrix forming agent can be used to impart crystallinity and hardness. Water can be used in the manufacturing process to ensure the production of porous units which disintegrate rapidly on the tongue. A preservative, such as a para-benzoic acid, at bacteriostatic concentration, can be used to prevent microbiological growth of the aqueous solution during the manufacturing process.

As used herein, the term “polymer” shall be understood to include the following: gelatins; modified starches; materials derived from animal or vegetable proteins; dextrins and soy; wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

A range of modified starches are commercially available and useful in the present invention and include:

Pregelatinized starches, produced by drum drying or extrusion;

Low-viscosity starches, produced by controlled hydrolysis of glycosidic bonds;

Dextrins, produced by roasting dry starch in the presence of a small amount of acid;

Acid modified starches, produced by suspension in dilute acid until the required viscosity is reached;

Oxidized starches, in which oxidizing agents cause the introduction of carbonyl or carboxyl groups, wherein depolymerization occurs, leading to decreased retrogradation and gelling capacities;

Enzymatically modified starch, produced by controlled enzyme degradation to attain required physicochemical properties;

Crosslinked starches, generated by reacting bi- or polyfunctional reagents (e.g., phosphorus oxychloride, sodium trimetaphosphate and epichlorohydrin) with hydroxyl groups to form crosslinks; and,

Stabilized starches, produced by reacting a starch with etherifying or esterifying reagents in the presence of an alkaline catalyst to give a wide range of products.

As used herein, the term “matrix forming agent” shall be understood to include include sugars such as mannitol, dextrose, lactose, galactose and trehalose; cyclic sugars such as cyclodextrin; inorganic salts such as sodium phosphate, sodium chloride and aluminum silicates; and amino acids having from 2 to 12 carbon atoms such as a glycine, L-alanine, L-aspartic acid, L-glutamic acid, L-hydroxyproline, L-isoleucine, L-leucine and L-phenylalanine.

One or more matrix forming agents may be incorporated into the solution or suspension prior to solidification. The matrix forming agent may be present in addition to a surfactant or to the exclusion of a surfactant. In addition to forming the matrix, the matrix forming agent may aid in maintaining the dispersion of any active ingredient within the solution, suspension or mixture. This is especially helpful in the case of active agents that are not sufficiently soluble in water and must, therefore, be suspended rather than dissolved.

Suspending or flocculating agents, or both, for example various gums, can be used to prevent the sedimentation of dispersed drug particles in the manufacturing process. pH-adjusting excipients, such as citric acid and sodium hydroxide, can be used to optimize the chemical stability of the drug, to minimize the solubility of water-insoluble compounds or to optimize the extent of ionization of drugs which are absorbed into the blood stream through the pregastric membranes. Permeation enhancers such as sodium lauryl sulphate can be used to optimize the transmucosal delivery of drugs absorbed through pre-gastric tissues. Collapse protectants such as glycine can be used to prevent the shrinkage of the units during the freeze-drying process or during long term storage. Flavors and sweeteners can be used to optimize taste, and microencapsulation polymers, such as various celluloses, can be used to mask any bitterness. Coloring agents can be used to impart product differentiation.

An example of a lyophilized formulation using sodium hydroxide as a pH-adjusting excipient is shown as Example 1.

### EXAMPLE 1

| | Concentration* | |
|---|---|---|
| Ingredient | % w/w | mg/unit |
| Compound A Bisulfate | 8 | 40 |
| Gelatin | 3.5 | 17.5 |
| Mannitol | 3 | 15 |
| Flavor (spearmint or peppermint) | 0.5 | 2.5 |
| Aspartame | 0.5 | 2.5 |
| 10% NaOH | 4 | 20 |
| Purified Water | q.s. to 100 mL | q.s. to 500 mL |
| Total | 100 | 500 |

*Expressed on a wet-weight, i.e., suspension basis, prior to lyophilization.

Prototypes of Example 1 displayed disintegration times of about 2 seconds and have acceptable stability. Essentially 100% of the Compound A Bisulfate dissolved within a 15-minute time frame when tested in an in vitro dissolution setting such as that referenced supra.

### Buffering Systems

The prototype formulations first prepared included NaOH as a pH adjusting excipient. The use of NaOH may be acceptable for initial pH adjustment of suspensions, however pH can subsequently drift over time due to the dissociation of the bisulfate salt into the free base and the counterion. Such pH variations can impact the performance of the final product. In order to stabilize pH, it was recognized that a buffer system with the appropriate buffering capacity is required. The purpose of the buffering system is to maintain the suspension pH at a suitable value, typically a pH of between about 3.5 and about 5.5, within the production time frame.

Pharmaceutically acceptable buffer systems can be used in conjunction with, or as an alternative to, the above-described pH-adjusting excipients. The selection of the buffer system is based on the targeted pH range, which in this case is between about 3.5 and about 5.5, preferably, between about 4 and about 5. These pH ranges are required to meet the stability requirement of Compound A Bisulfate and the performance of the rapidly disintegrating solid dosage form. In particular, it was found that lower pHs can adversely impact on the dissolution rate of the final product. Pharmaceutically acceptable buffer systems capable of maintaining pH in these ranges include acetate, phosphate, and citrate buffer systems. Examples of such buffer systems include acetic acid/sodium acetate, phosphoric acid/sodium phosphate, and citric acid/sodium citrate systems. Additional buffer systems within the scope of the present invention include those based on the following water soluble acids and their salts:

(+)-L-Tartaric acid;
D-Glucuronic acid;

### Glycolic Acid;

### D-Glucoheptonic Acid;

### (−)-L-Pyroglutamic Acid;

### DL-Mandelic Acid;

### (−)-L-Malic Acid;

### Formic Acid;

### D-Gluconic Acid;

### DL-Lactic Acid;

### L-Ascorbic Acid;

Succinic Acid; and,

### Glutaric Acid.

In order to determine the concentrations of buffer system components for a specific pre-lyophilized TRA suspension, a series of citrate buffer systems for Compound A bisulfate suspensions was prepared for various target pHs according to Table 1A below:

TABLE 1

| Buffer | | Concentration | [Citric Acid] | [Sodium Citrate] |
|---|---|---|---|---|
| No. | pH | (mM) | (g/L) | (g/L) |
| 1 | 3 | 50 | 8.98 | 2.13 |
| 2 | 3 | 100 | 17.96 | 4.26 |
| 3 | 3 | 200 | 35.93 | 8.53 |
| 4 | 4 | 50 | 6.56 | 5.53 |
| 5 | 4 | 100 | 13.12 | 11.05 |
| 6 | 4 | 200 | 26.23 | 22.10 |
| 7 | 5 | 50 | 3.85 | 9.32 |
| 8 | 5 | 100 | 7.69 | 18.64 |
| 9 | 5 | 200 | 15.38 | 37.29 |

Mt. of citric acid = 210.0
Mt. of sodium citrate = 291.4

To a 20 ml aliquot of each buffer solution of Table 1 was added 0.7 g of gelatin. The solutions were heated to 60° C. (via hotplate w/stirring) for approximately 30 minutes. Then, the solutions were cooled to ˜25° C. (via water bath) and pH was recorded. 1.6 g of Compound A bisulfate (the active pharmaceutical ingredient or “API”) was added to solutions to prepare an 8 w/w % suspension. During sample preparation, about ⅓^{rd} of the solution was added to prepare a survey. Each survey was homogenized to ensure homogeneity. The remaining ⅔^{rd} of the solution was then added while stirring. Each suspension was held for 48 hours and pH was recorded. The results of the pH measurements are shown in Table 2/3.

TABLE 2

| | | | | | | pH |
|---|---|---|---|---|---|---|
| | Buffer | Tar- | Ac- | pH after | pH after | after 48 |
| Buffer | Solution | get | tual | Gelatin | API | Hour |
| No. | Concentration | pH | pH | Addition | Addition | Hold |
| 1 | 50 mM | 3 | 2.88 | 3.37 | 2.88 | 1.31 |
| 2 | 100 mM | 3 | 2.87 | 3.12 | 2.79 | 1.16 |
| 3 | 200 mM | 3 | 2.84 | 2.93 | 2.77 | 2.32 |
| 4 | 50 mM | 4 | 3.82 | 4.12 | 3.62 | 2.65 |
| 5 | 100 mM | 4 | 3.84 | 3.97 | 3.77 | 2.83 |
| 6 | 200 mM | 4 | 3.80 | 3.84 | 3.75 | 3.53 |
| 7 | 50 mM | 5 | 4.84 | 5.04 | 3.97 | 3.61 |
| 8 | 100 mM | 5 | 4.80 | 4.90 | 4.38 | 3.83 |
| 9 | 200 mM | 5 | 4.72 | 4.72 | 4.76 | 4.28 |

The experimental data show that for a citrate buffer (in this case, a citric acid/sodium citrate buffer system) the systems that target pHs of 4 and 5 at any of the concentrations of 50, 100 and 200 mM (in this case, Buffer Nos. 4-9) are adequate to control Compound A Bisulfate suspension pH between 3.5 and 5.5, as measured after API addition. This will be adequate in most process scenarios, in which lyophilization is occurs directly after suspension preparation. However, in cases in which the suspension is stored for up to 48 hours prior to lyophilization, only the systems that target a pH of 5 at concentrations of 50, 100, and 200 mM (Buffer Nos. 7-9), and the system that targets a pH of 4 at a concentration of 200 mM (Buffer No. 6) are adequate to maintain the suspension pH within the range of 3.5-5.5.

To maintain a pH of 5, citric acid concentrations of 0.7 to 3.4% w/w and sodium citrate concentrations of 1.7 to 8.3% w/w were found to be effective. To maintain a pH of 4, citric acid concentrations of 1.2 to 5.8% w/w and sodium citrate concentrations of 1.0 to 4.9% w/w were found to be effective. Thus, to maintain a pH range of about 4 to about 5, a citric acid concentration range of about 0.7 to about 5.8% w/w and a sodium citrate concentration range of about 1.0 to about 8.2% w/w would be expected to be effective. The concentrations of citric acid and sodium citrate are expressed with respect to the pre-lyophilized suspension.

Based on the above results, a lyophilized formulation of Compound A Bisulfate can be buffered as shown in Example 2.

### EXAMPLE 2

| | Concentration* | |
|---|---|---|
| Ingredient | % w/w | mg/unit |
| Compound A Bisulfate | 8 | 40 |
| Gelatin | 3.5 | 17.5 |
| Mannitol | 3 | 15 |
| Flavor (spearmint or peppermint) | 0.5 | 2.5 |
| Aspartame | 0.5 | 2.5 |
| Sodium Citrate | 3.73 | 18.65 |
| Citric Acid | 1.54 | 7.70 |
| Purified Water | q.s. to 100 mL | q.s. to 500 mL |
| Total | 100 | 500 |

*Expressed on a wet-weight, i.e., suspension basis, prior to lyophilization.

Based on the above, it is believed that useful lyophilized formulations of a 40 mg loading dose of Compound A, or its pharmaceutically acceptable salts and hydrates, include those comprising:
gelatin in an amount of about 16 to about 19 mg, preferably about 17.5 mg;
mannitol in an amount of about 14 to about 16 mg, preferably about 15 mg;
sodium citrate in an amount of about 18 to about 19 mg, preferably about 18.7 mg; and,
citric acid in an amount of about 7 to about 8 mg, preferably about 7.7 mg.

Alternate embodiments in which the excipient components recited in Examples 1 and 2 above are substituted with other components within the same functional class are within the scope of the present invention. Thus, embodiments in which gelatin is substituted with another polymer, e.g., starch, are encompassed in the present invention. Similarly, embodiments in which mannitol is substituted with another matrix forming agent, e.g., lactose, are encompassed in the present invention. Flavoring agents (e.g., spearmint) and sweetening agents (e.g., aspartame) are also substitutable within class. As discussed above, buffering systems can be substituted.

Furthermore, the wet-basis (i.e., pre-lyophilized) percent compositions (under the heading “% w/w”) disclosed in Examples 1 and 2 are exemplary, but not limiting. For example, the applicable range of wet-basis compositions of polymer (e.g., gelatin) is from about 2 to about 5% w/w, and that of matrix forming agent is from about 2 to about 4% w/w. The concentrations of flavoring agents and of sweetening agents can be varied as required. As discussed above, concentrations of the components of the buffering systems can be varied to some degree, while maintaining desired pH.

Higher doses of Compound A can be incorporated into the inventive formulations. Loading doses of up to 150 mg are within the scope of the present invention. For example, loading doses of 80 and of 120 mg of Compound A, or of a pharmaceutically acceptable salt or hydrate thereof, are within the scope of the present invention.

### Lyophilization Process

In essence, lyophilization (i.e., freeze-drying) consists of at least two steps: first, freezing a solution or suspension (almost invariably aqueous) of the material to be freeze-dried; and second, raising the temperature of the frozen material with a concomitant application of vacuum so that the frozen solvent (almost invariably ice) sublimes without melting. The term “lyophilized” as used herein will be understood to refer to a formulation that is the product of at least these two processing steps. The effects of freeze-drying temperature on formulation appearance and processing times are discussed in U.S. Pat. No. 5,044,091, which is herein incorporated in relevant part.

The production sequence typically begins with the bulk preparation of an aqueous drug solution or suspension and subsequent precise dosing into pre-formed blisters. It is the blister that actually forms the tablet shape and is, therefore, an integral component of the total product package. The second phase of manufacturing typically entails passing the filled blisters through a specially designed cryogenic freezing process to control the ultimate size of the ice crystals. This aids in ensuring that the tablet possesses a porous matrix to facilitate the rapid disintegration function. These frozen units are then transferred to large-scale freeze dryers for the sublimation process, whereby the majority of the remaining moisture is removed from the tablets. The final phase of production involves sealing the open blisters via a heat-seal process to ensure stability and to protect the product from varying environmental conditions. Procedures for preparing formulations in this aspect of the present invention are described in, for example, U.S. Pat. Nos. 6,509,040 and 6,709,669, both of which are hereby incorporated by reference.

An example of a commercially available freeze-drying technology applicable to such dosage forms is known by its trade name of Zydis®, and is available from Catalent, formerly Cardinal Health, of Somerset, N.J. See H. Sager, “Drug-delivery Products and the Zydis Fast-dissolving Dosage Form,” J. Pharm. Pharmacol. 50:375-382 (1998). As an example of such a product, a freeze-dried formulation of olanzapine is marketed by Eli Lilly as Zyprexa® Zydis® orally disintegrating tablets. Inactive ingredients include gelatin, mannitol, asparatame, sodium methyl paraben and sodium propyl paraben. Claritin® RediTabs®, marketed by Schering-Plough Corp., provides another example of Zydis-based formulations, which are as follows:

TABLE 3

| Ingredient | Amt (mg) | Wt %** | Amt (mg) | Wt %** |
|---|---|---|---|---|
| Loratadine | 5 | 23.2 | 10 | 37.6 |
| Gelatin NF | 8.985 | 41.7 | 8.985 | 33.8 |
| Mannitol USP | 7.188 | 33.3 | 7.188 | 27.1 |
| Flavor Mint 51296 TP0551 | 0.150 | 0.7 | 0.150 | 0.6 |
| Anhydrous Citric Acid | 0.250 | 1.2 | 0.250 | 0.9 |
| USP | | | | |
| Purified Water | (—)* | (—)* | (—)* | (—)* |
| Theoretical Dry Tablet | 21.573 | 100% | 26.573 | 100% |

*Sublimed during lyophilization.
**Dry Basis

### Acute Coronary Syndrome

The present invention further encompasses methods of treatment of a patient at risk of Acute Coronary Syndrome by administering an effective amount of a rapidly disintegrating formulation of a thrombin receptor antagonist as described above. As used herein, the term “effective amount” will be understood to describe an amount of a thrombin receptor antagonist effective to prevent further damage to the cardiovascular system after an acute cardiac event.

In the treatment of ACS, thrombin receptor antagonist dosing regimens will comprise one-time administration of a loading dose followed by regular administration of maintenance doses. The TRA concentration of the loading dose will be sufficient to very quickly achieve high levels of platelet aggregation inhibition. Following administration of the loading dose, platelet aggregation inhibition levels of at least 80-90% within no more than 1-2 hours are targeted. TRA concentrations in the loading dose formulation will be 20-120 mg. The TRA concentration of the maintenance dose will be sufficient to maintain the desired levels of platelet aggregation inhibition. TRA concentrations in the maintenance dose formulation will be 1-10 mg. A dosing regimen of the bisulfate salt of Compound A comprising a 40 mg loading dose followed by daily 2.5 mg maintenance doses is planned for evaluation in phase iii clinical trials. The rapidly disintegrating solid dosage forms of the present invention are intended for administration as loading doses within these dosing regimens. Such lyophilized loading dose formulations may be capable of attaining platelet aggregation inhibition of at least about 80% within 30 minutes of administration. They may allow essentially all of the thrombin receptor antagonist to dissolve within about 15 minutes.

The lyophilized TRA formulations of the present invention will also find utility in treating victims of acute stroke and patients who undergo percutaneous coronary intervention (“PCI”).

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

What is claimed is:

1. A lyophilized rapidly disintegrating solid dosage form comprising an effective amount of a thrombin receptor antagonist.

2. The rapidly disintegrating solid form according to claim 1 wherein the thrombin receptor antagonist is selected from the group consisting of: 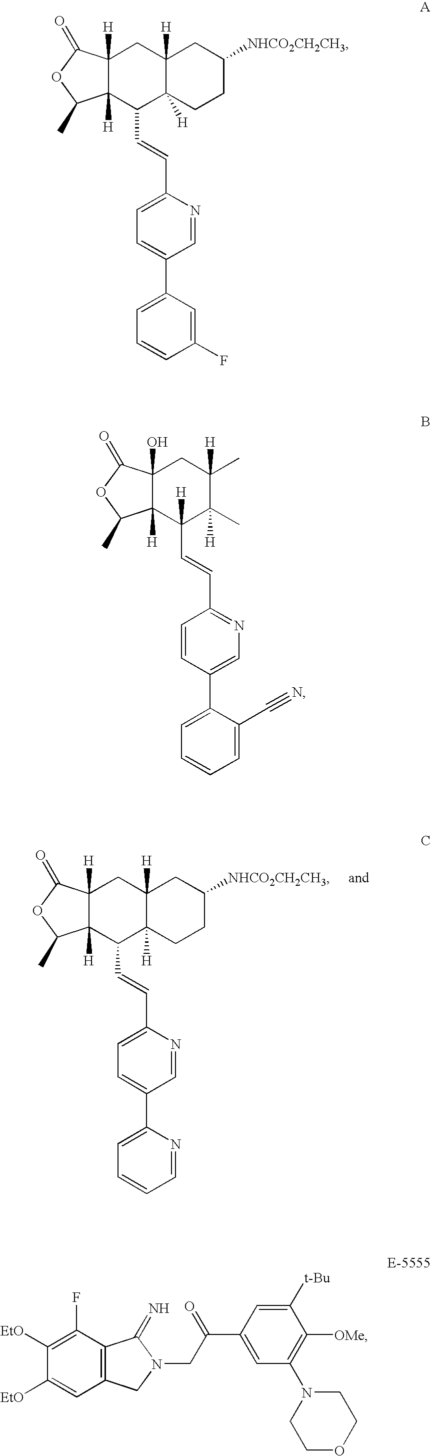
or a pharmaceutically acceptable salt or hydrate thereof.

3. The rapidly disintegrating solid dosage form according to claim 2, further comprising at least one polymer and at least one matrix forming agent.

4. The rapidly disintegrating solid dosage form according to claim 3, wherein the polymer is selected from the group consisting of gelatin, alginates, and modified starches.

5. The rapidly disintegrating dosage form according to claim 3, wherein the matrix forming agent is selected from the group consisting of mannitol, sorbitol, and dextrins.

6. The rapidly disintegrating dosage form according to claim 1, further comprising a buffering system.

7. The rapidly disintegrating dosage form according to claim 6 wherein said buffering system is selected from the group consisting of acetate, phosphate, and citrate buffer systems.

8. The rapidly disintegrating solid dosage form according to claim 1 wherein an average platelet inhibition of at least about 80% is achieved within 30 minutes of administration.

9. A lyophilized rapidly disintegrating solid dosage form comprising about 20 to about 120 mg of Compound A 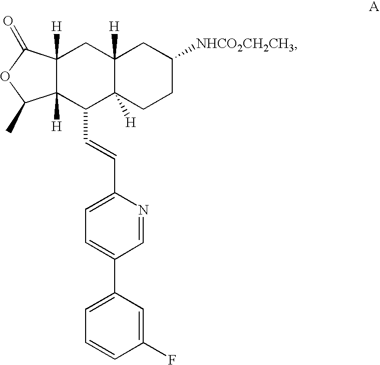
or a pharmaceutically acceptable salt or hydrate thereof.

10. The rapidly disintegrating solid dosage form according to claim 9 comprising about 40 mg of Compound A or a pharmaceutically acceptable salt or hydrate thereof.

11. The rapidly disintegrating solid dosage form according to claim 9, wherein Compound A is in the form of a bisulfate salt.

12. A lyophilized rapidly disintegrating solid dosage form comprising about 40 mg of Compound A or a pharmaceutically acceptable salt or hydrate thereof, a polymer, and a matrix forming agent.

13. The rapidly disintegrating solid dosage form according to claim 12, further comprising a buffer system.

14. A method of treating a patient at risk of acute coronary syndrome comprising administering to said patient a single lyophilized loading dose comprising an effective amount of a thrombin receptor antagonist and then a series of maintenance doses comprising said thrombin receptor antagonist.

15. The method according to claim 14 wherein said thrombin receptor antagonist is selected from the group consisting of: 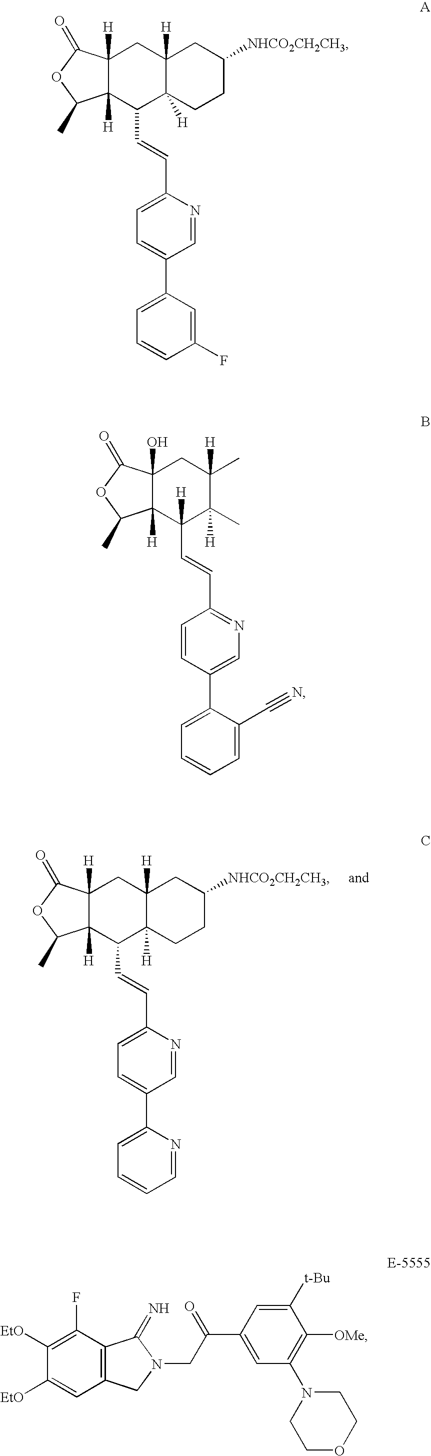
or a pharmaceutically acceptable salt or hydrate thereof.

16. The method according to claim 14 wherein said thrombin receptor antagonist is 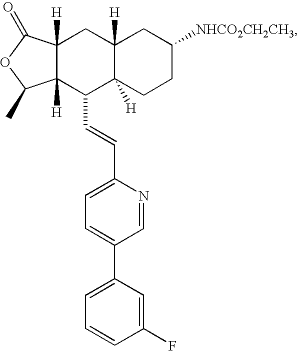
or a pharmaceutically acceptable salt or hydrate thereof.

17. The method according to claim 16 wherein said lyophilized loading dose comprises between about 20 and about 120 mg of said thrombin receptor antagonist.

18. The method according to claim 16 wherein said lyophilized loading dose comprises about 40 mg of said thrombin receptor antagonist.

19. The method according to claim 16, wherein said thrombin receptor antagonist is in the form of a bisulfate salt.
